(19) United States
(12) Patent Application Publication
(10) Pub. No.: US 2020/0043061 A1
(43) Pub. Date: Feb. 6, 2020

(54) WELLNESS THROUGH FAVOR MONITORING

(71) Applicant: INTERNATIONAL BUSINESS MACHINES CORPORATION, ARMONK, NY (US)

(72) Inventor: Jessika A. Welch, Amesbury, MA (US)

(21) Appl. No.: 16/050,839

(22) Filed: Jul. 31, 2018

(51) Int. Cl. G06Q 30/02; G16H 50/30; G16H 10/60

(52) U.S. Cl. CPC G06Q 30/0279; G06Q 50/01; G16H 10/60; G16H 50/30

(57) ABSTRACT

A method, computer program product, and system for favor monitoring and wellness monitoring, the method including sharing a first favor request from a first user to a first group of one or more users, wherein the favor request comprises, a favor type, a favor time request start, a favor time request end, and one or more favor parameters, receiving a first favor proposal from a second user of the first group, receiving a first favor acknowledgement from the first user in response to the first favor proposal, receiving confirmation of completion of the first favor, documenting completion of the first favor, wherein documentation comprises the first favor request, the first user, and the second user, monitoring a favor request history of a third user of the first group, and identifying the third user as an at risk user based on a change greater than a first threshold amount of a number of favor requests over a period of time, and based on a percentage of unfulfilled requests greater than a second threshold amount.

### BACKGROUND

The present invention relates generally to a method, system, and computer program product for information processing, and more particularly for wellness monitoring through a favor application.

Acts of kindness bring happiness to both the recipient and the giver of help. Family and friends often are willing and want to help, but often are unaware of the needs of others. For many of us, it is difficult to ask for help or for a favor. Often people are more comfortable assisting others rather than requesting help. Asking for help can deepen a relationship. This is known as The Benjamin Franklin Effect, named after statesman Benjamin Franklin, who proclaimed, ‘He that has once done you a kindness will be more ready to do you another than he whom you yourself have obliged.’ Fostering an environment for people to help each other can provide short-term solutions to individuals, and also long-term benefits to our community and society. Many problems and situations can be solved through favors, and the benefits to both recipient and giver are hugely positive.

### SUMMARY

Embodiments of the present invention disclose a method, computer program product, and system for favor monitoring and wellness monitoring, the method including sharing a first favor request from a first user to a first group of one or more users, wherein the favor request comprises, a favor type, a favor time request start, a favor time request end, and one or more favor parameters, receiving a first favor proposal from a second user of the first group, receiving a first favor acknowledgement from the first user in response to the first favor proposal, receiving confirmation of completion of the first favor, documenting completion of the first favor, wherein documentation includes the first favor request, the first user, and the second user, monitoring a favor request history of a third user of the first group, and identifying the third user as an at risk user based on a change greater than a first threshold amount of a number of favor requests over a period of time, and based on a percentage of unfulfilled requests greater than a second threshold amount.

### BRIEF DESCRIPTION OF THE DRAWINGS

The following detailed description, given by way of example and not intended to limit the invention solely thereto, will best be appreciated in conjunction with the accompanying drawings, in which:

FIG. 1 is a functional block diagram illustrating a distributed data processing environment, in accordance with an embodiment of the present invention;

FIG. 2 is a flowchart depicting operational steps of a favor and wellness flowchart within the data processing environment of FIG. 1, in accordance with an embodiment of the present invention;

FIG. 3 is a block diagram depicting operational steps of a monitoring program within the data processing environment of FIG. 1, shown in accordance with an embodiment of the present invention;

FIG. 4 is a block diagram of internal and external components of computers and servers depicted in FIG. 1, in accordance with an embodiment of the present invention;

FIG. 5 is a block diagram of functional layers of an illustrative cloud computing environment, including the distributed data processing environment depicted in FIG. 1, in accordance with an embodiment of the present disclosure; and

FIG. 6 is a functional block diagram of functional layers of the illustrative cloud computing environment of FIG. 5, in accordance with an embodiment of the present invention.

The drawings are not necessarily to scale. The drawings are merely schematic representations, not intended to portray specific parameters of the invention. The drawings are intended to depict only typical embodiments of the invention. In the drawings, like numbering represents like elements.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the interest of not obscuring the presentation of embodiments of the present invention, in the following detailed description, some processing steps or operations that are known in the art may have been combined together for presentation and for illustration purposes and in some instances may have not been described in detail. In other instances, some processing steps or operations that are known in the art may not be described at all. It should be understood that the following description is rather focused on the distinctive features or elements of various embodiments of the present invention.

Embodiments of the present invention relate to the field of information processing, and more particularly to providing wellness monitoring through a favor application. The following described exemplary embodiments provide a system, method, and program product to, among other things, monitor favor requests and favor fulfillment, and also help identify people with low or high favor requests, a significant change in quantity or type of favor requests, and unfulfilled favor requests or combination thereof, in order to identify people who may need help, as well as identify those who may provide help. Therefore, the present embodiment has the capacity to help monitor wellness of users registered in the favor application.

Busy families are often stretched timewise to complete circumstantial tasks that conflict with daily schedule or routine. There may be stress in trying to find help in completing some necessary tasks. Paid services are sometimes the answer, for example a ride sharing application, a delivered meal, or hiring a sitter, but stress may remain with doubts about the service provider chosen to complete the task. Doubts may include: ‘Will the assigned task be completed and completed accurately?’ and ‘Will my dependents be safe?’. People cannot be in two places at once, and choosing one place over another often results in disappointment. For example, unable to attend the job interview because there is no one to watch the kids, or guilt, such as, a child being unable to attend a sports practice due the absence of transportation. The stress of completing out of the ordinary tasks is not limited to families. Individuals, too, find difficulty in completing tasks due to the limitations of their situation. A non-driver may have trouble running errands in advance of a storm, or getting to the polls to vote. Asking for help, especially from the same friend repeatedly, can make one feel like a burden to others. The relationship may get strained. Asking for favors may bring up deep-seated fears and feelings of awkwardness. Fear of rejection, appearing weak, being an inconvenience, appearance of using another person, owing a debt to another person, or losing social capital, which could have been used for something else, may contribute to an individual's adverseness to asking for a favor.

Acts of kindness can bring happiness to the performers. Family and friends often want to help each other and frequently may be available to help, but may be unaware of the needs of people in their network. A virtual place or application can be used where people can both ask for and receive help from friends and family without awkwardness. People can post a favor request, which states what is needed and why, where, and when the favor is needed, and select to whom you would like to send the favor request to. People can also communicate how and in what ways they can help through completing a favor profile. The application may include an area to engage offers, accept or propose alternative offers of help, and determine reciprocity for the favor, if desired, for example a favor in return or time-on-credit. An equitable arrangement, such as a favor for a favor, specific or at a future, yet undetermined date, can bring purpose and relief to both parties.

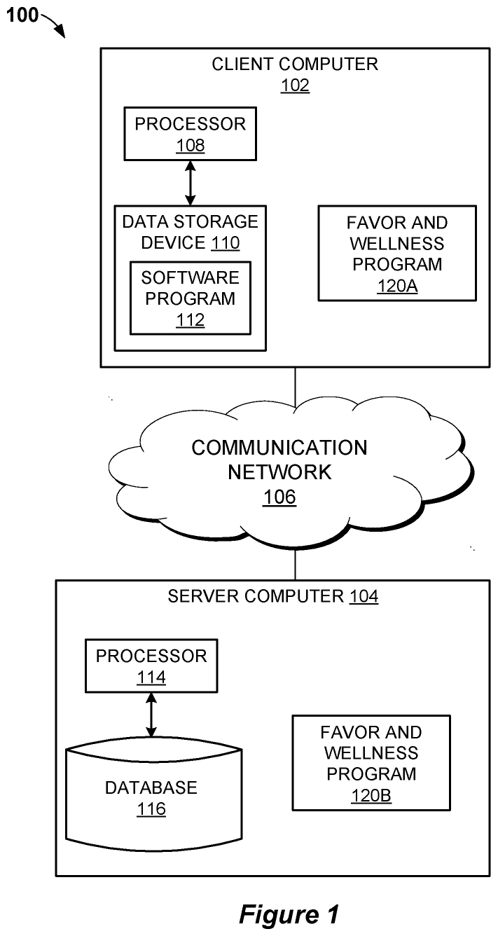

Referring now to FIG. 1, a functional block diagram illustrating a system 100 in a networked computer environment, in accordance with an embodiment of the present invention, is shown. The system 100 may include a client computer 102 and a server computer 104. The client computer 102 may communicate with the server computer 104 via a communication network 106 (hereinafter “network”). The client computer 102 may include a processor 108, and a data storage device 110 that is enabled to host and run a software program 112 and a favor and wellness program 120A. The client computer 102 is enabled to interface with a user and communicate with the server computer 104. The server computer 104 may also include a processor 114 and a database 116 that is enabled to run a favor and wellness program 120B. In an embodiment, the client computer 102 may operate as an input device including a user interface while the favor and wellness program 120B runs primarily on the server computer 104. In an alternative embodiment, the favor and wellness program 120A may run primarily on the client computer 102 while the server computer 104 may be used for processing a storage of data used by the favor and wellness program 120A.

It should be noted, however, that processing for the favor and wellness program 120A, 120B may, in some instances be shared amongst the client computer 102 and the server computer 104 in any ratio. In another embodiment, the favor and wellness program 120A, 120B may operate on more than one server computer 104, client computer 102, or some combination of server computers 104 and client computers 102, for example, a plurality of client computers 102 communicating across the network 106 with a single server computer 104.

The network 106 may include wired connections, wireless connections, fiber optic connections, or some combination thereof. In general, the network 106 can be any combination of connections and protocols that will support communications between the client computer 102 and the server computer 104. The network 106 may include various types of networks, such as, for example, a local area network (LAN), a wide area network (WAN) such as the Internet, a telecommunication network, a wireless network, a public switched network and/or a satellite network.

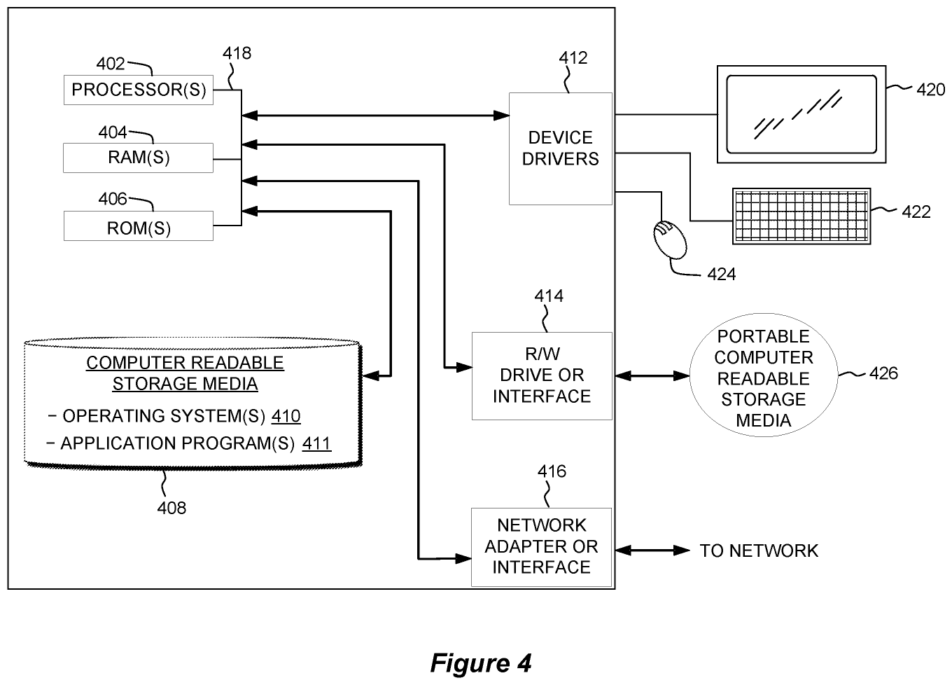

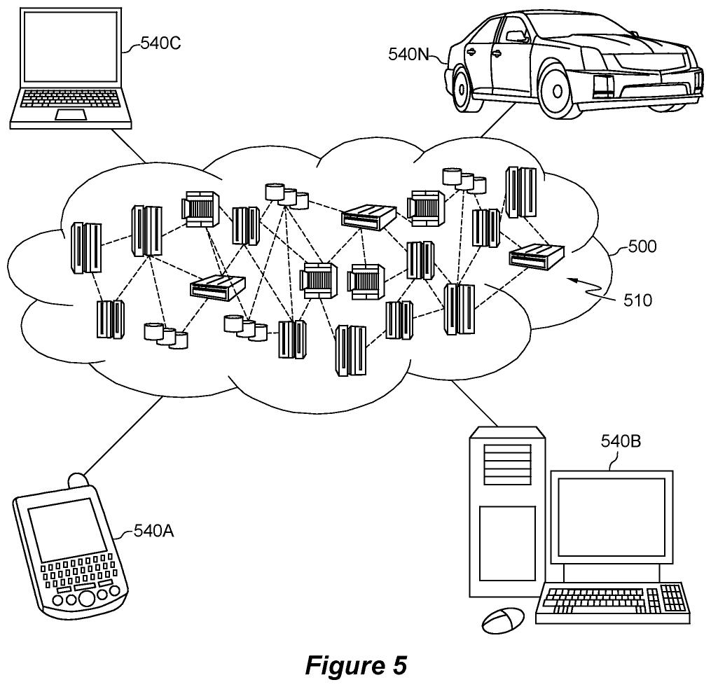

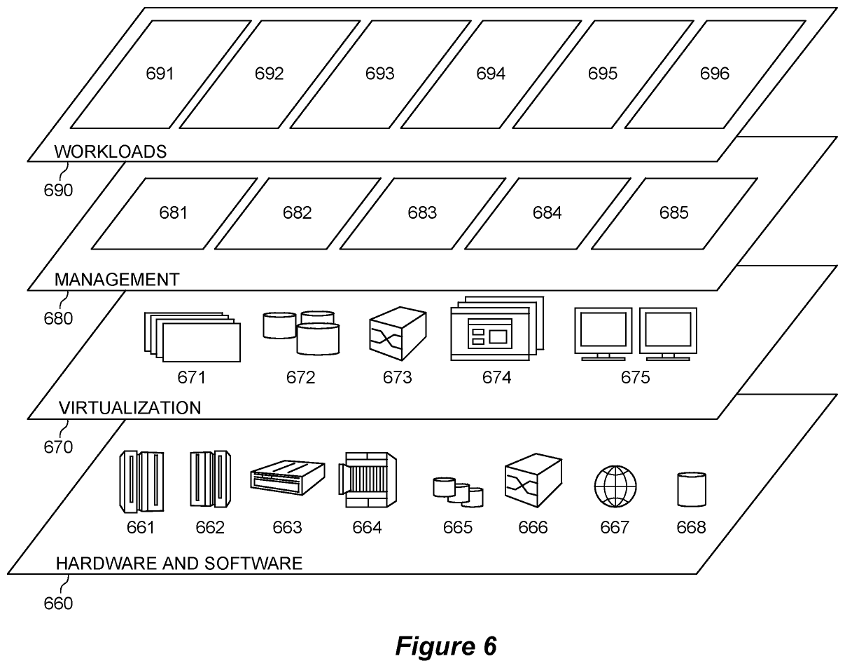

In various embodiments, the client computer 102 and/or the server computer 104 may be, for example, a laptop computer, tablet computer, netbook computer, personal computer (PC), a desktop computer, a personal digital assistant (PDA), a smart phone, a mobile device, or any programmable electronic device capable of communicating with the server computer 104 via the network 106. As described below with reference to FIG. 4, the client computer 102 and the server computer 104 may each include internal and external components. In other embodiments, the server computer 104 may be implemented in a cloud computing environment, for example, cloud computing nodes 510, as described in relation to FIGS. 5 and 6 below. Similarly, the client computer 102 may be implemented in the cloud computing environment, for example, laptop computer 540C as shown in FIG. 5.

In an embodiment, the system 100 may include any number of client computers 102 and/or server computers 104; however only one of each is shown for illustrative purposes only. It may be appreciated that FIG. 1 provides only an illustration of an implementation and does not imply any limitations with regard to the environments in which different embodiments may be implemented. Many modifications to the depicted environments may be made based on design and implementation requirements.

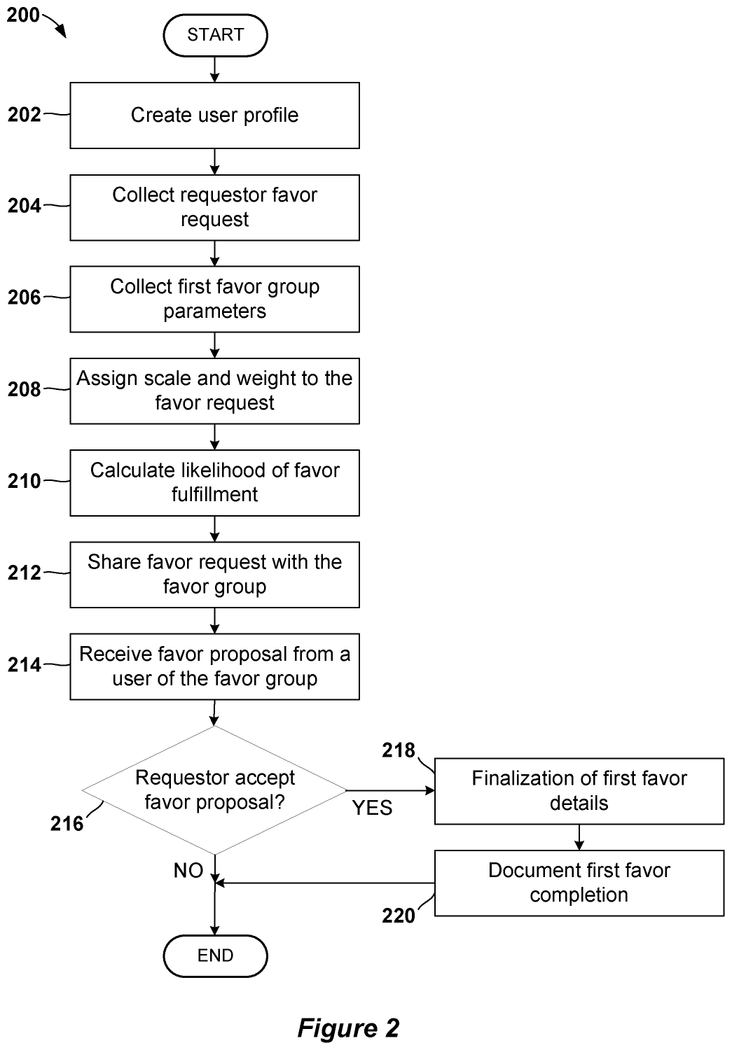

According to an embodiment, the favor and wellness program 120A, 120B may be generally configured to collect user information, collect a favor request, help coordinate matching of a favor requestor and a favor granter, collect a favor fulfillment, and monitor wellness of a user. The favor and wellness method is explained in further detail below with respect to FIGS. 2-6.

Referring now to FIG. 2, and with continued reference to FIG. 1, a favor and wellness flowchart 200 is shown in accordance with an embodiment of the present invention. The favor and wellness flowchart 200 may be used to manage a favor request and track responses and other actions related to the favor request.

Beginning with step 202, a user profile is created by a user. There may be two or more user profiles. The user profile may include a userid, a user name, an email of the user, an address of the user, a phone number of the user, and additional contact information of the user. The user may provide or share a calendar which identifies free time that the user may be available to assist in helping other users with a favor request. A first user of the two or more user profiles may identify a second user which is a user profile of a person whom they know, or have something in common with, for example, they are both members of a community living in close proximity, they share a place of employment, or other association. The first user may issue a friend request to the second user and other users in the application. The second user may accept the friend request and the first user and the second user would then be connected and able to see information about each other.

The user profile may identify the types of favor requests that the user is willing to provide help for and willing to or generally wants to receive help for. Types of favor requests may include providing transportation, childcare, dog walking or sitting, house sitting, picking up items, yard work, house work, borrow something, advice, need a place to stay, home maintenance, car maintenance, recommendations, and other activities. Additionally, the user profile may specify types or sub-types of requests that they are not available to perform, for example, they are available for dog walking, and are willing to watch a single dog, under 30 pounds, but not multiple dogs, nor a dog over 30 pounds. The user profile may identify a group of one or more friends amongst other users in the application whom they would be willing to assist in performing help or provide a favor for. The user profile may identify a group of one or more acquaintances amongst other users in the application whom they would like to include when they send out their own favor requests. The user profile may include a group of one or more coworkers amongst other users in the application whom they would include for work related favor requests or to provide assistance for when a favor request is made.

At step 204, the first user creates a first favor request. The first favor request may identify a type of favor, specify the first favor details such as what is needed, a time or a time range requested for the first favor to be performed, and a location of where the first favor is to be performed, among other first favor details. The favor request may identify why the first favor is needed. For example, the first favor request may include the type of the first favor is ‘dog walking’, the first favor details may include ‘please walk my dog Cosmo, he is an 8-year-old, 20 pound dog’, the time requested may include ‘on Monday, September 10, between 2 pm and 4 pm, and the first favor location may include an address. The first favor request may indicate ‘my usual dog walker will be unavailable on that date’.

At step 206, the first user identifies a first favor group to whom the first favor request would be shared with or assign parameters of the first favor group to be selected from a group of friends of the first user. The first user may select one of their original groups from the creation of the first user profile, i.e. the group of one or more friends, the group of one or more acquaintances, the group of one or more coworkers, or another group. Alternatively, the first user may create a new group, or edit one of their existing groups. In an alternate embodiment, the first user may select users within a geographic area, for example, those users who live on the same street as the first user, to ask them to water their flowers while they are away.

At step 208, favor and wellness program 120A, 120B assigns a scale and weight to the favor request. A scale of the favor request may indicate a length of time or an amount of money expended in satisfying the favor request. For example, borrowing an item for a day may have a lower scale than borrowing an item for a week. Alternatively, providing transportation for 30 minutes may have a lower scale than providing transportation for an hour. A weight of the favor request may indicate a complexity of the favor request, with a higher weight identifying a more complex favor. For example, borrowing an item may have a low complexity or weight of 2 out of 10 and providing transportation may have a higher complexity or weight of 7 out of 10. The total weight applied for each favor may be configured by a user. For example, a user may add a higher weight for dog sitting than for dog walking since dog sitting requires more time and effort to complete.

In an embodiment, a favor weight may be low, medium, high, emergency, and each of these favor weights may be assigned a value, for example 1, 2, 3, and 4, respectively.

The favor and wellness program 120A, 120B may track favor exchanges and allow the first user to provide feedback on favor fulfillment performance by another user. Fulfillment exchanges may occur between two users, but the details may be shared and available to multiple users. The fulfiller of the favor may receive a credit to their account and the receiver of the favor may be debited an equivalent amount. In an embodiment, the credit or debit may be calculated as a multiplication of the scale and weight of the favor. The favor and wellness program 120A, 120B may store and display the user's transaction history and provide a summary which includes a favor balance and feedback summary. Relationships between different users may be defined by their exchange history.

At step 210, the favor and wellness program 120A, 120B calculates a likelihood of a first favor request fulfillment. The first favor request fulfillment likelihood may take into account specifics of the first favor request, for example, the type of favor request, the requested time of the favor request, and a duration of the favor request, and compare the specifics of the favor request with the first favor group, and the specifics of what the users of the first favor group have each specified as the type of favors they are willing to provide and their calendar of available free time. In an embodiment, the likelihood of the first favor request fulfillment may be a percentage between 0 and 100%. Based on the likelihood calculation, for example below a first threshold, the first user may modify their first favor request, for example, change the time requested for the first favor request to increase the likelihood of first favor request fulfillment. In an embodiment, the favor and wellness program 120A, 120B may provide suggested modifications to the first favor request in order to improve the likelihood of first favor request fulfillment. For example, the first favor request may be “please walk my dog Cosmo, he is an 8-year-old, 20 pound dog, on Monday, September 10, between 2 pm and 4 pm”. An original likelihood of the first favor request fulfillment may be 50%, and the first threshold may be 60%, so the original likelihood is lower than the first threshold. The favor and wellness program 120A, 120B may provide a suggestion that the first favor request be modified to a time range between 3 pm and 5 pm, which may result in an updated likelihood of the first favor request fulfillment increasing to 70%.

At step 212, the first user shares the first favor request with the first favor group. The one or more users in the first favor group may be able to see the details of the first favor request and contact information for the first user. In an embodiment, the first favor request is shared amongst a first subset of users in the first favor group whom indicated that they are willing to perform the favor type of the first favor, and have corresponding free time on their calendar that matches the first favor request, and the first favor request is not shared with a second subset of users in the first favor group whom either would not be willing to perform the favor type of the first favor or have a conflict during the requested time of the first favor. In an embodiment, the first favor request is sent to all of the users in the first favor group.

At step 214, a second user of the group of two or more users responds to the first favor request with a first favor proposal. The first favor proposal may include the contact information of the second user, and may include comments or questions from the second user. For example, the first favor request may be “please walk my dog Cosmo, he is an 8-year-old, 20 pound dog, on Monday September 10 between 2 pm and 4 pm”. The first favor proposal may include the message “I would be able to walk Cosmo at 1:30, is that ok? Please send your apartment number as well”.

At step 216, the favor and wellness program 120A, 120B determines whether the requestor has accepted the proposal. The first user, the requestor, may accept the first favor proposal through interactions with a peripheral device or a touchscreen. Alternatively, the first user may decline the first favor proposal and may send a message to the second user thanking them for their offer of help. In this situation, the first favor may remain in an unfulfilled state and alternate users of the first favor group may respond to the first favor request. Once the first user accepts an offer from one of the users of the first favor group, the first favor may have a status of favor offer accepted. Once the first favor is completed the first favor may have a status of favor requested completed. If a time requested for the favor has passed without fulfillment, the first favor may have a status of favor request expired. The first favor request status may be unfulfilled, accepted, completed, expired, or removed by the first user. If the favor and wellness program 120A, 120B determines the requestor has accepted the proposal (step 216, “Yes” branch), the favor and wellness flowchart 200 may continue to step 218 to finalize the first favor details. If the favor and wellness program 120A, 120B determines the request has declined the proposal (step 216, “No” branch), the favor and wellness program 120A, 120B may terminate.

At step 218, the first user and the second user finalize details of the first favor request. The finalization details may include final directions, acknowledgment of a weight and value of the first favor, and other information. The finalization may include barter details, for example money to be paid for the favor, or a second favor performed by the first user for the second user, or a credit and a debit, respectively, to the second user and first user favor accounts. The finalization details may be transmitted between the first user and the second user through a series of messages where each user provides requested information. In at least one embodiment, the finalization details may be provided automatically upon acceptance of the favor proposal.

At step 220, the favor and wellness program 120A, 120B collects documentation of competition of the first favor. The documentation may include confirmation that the favor was performed, and other details, for example, the first favor took 2 hours for completion rather than the 1.5 hours that were estimated. The documentation could include feedback of the first user by the second user, and feedback of the second user by the first user. For example, the first user could document that the second user performed the first favor well and was timely in their assistance. The documentation could be marked private or may be shared amongst a subset of other users.

The favor and wellness program 120A, 120B, may retain information and collect further information for future use. Information collected may be used to calculate favor success rate, help provide favor recommendations, insights, calculate a rating for user likelihood to help, etc.

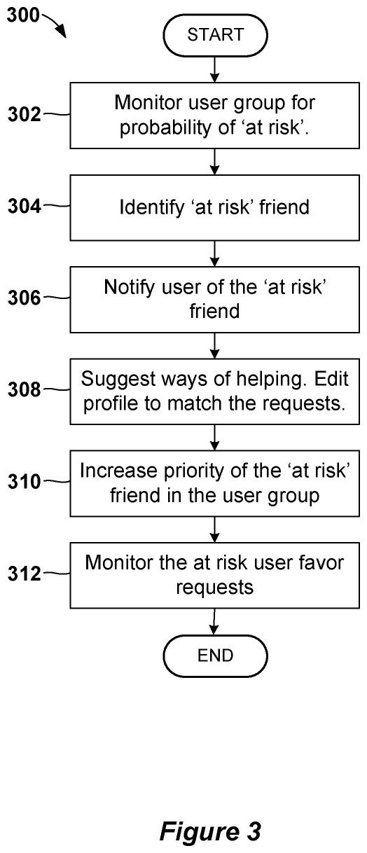

Referring now to FIG. 3, and with continued reference to FIGS. 1 and 2, a monitoring flowchart 300 is shown in accordance with an embodiment of the present invention. The monitoring flowchart 300 may use information collected from the favor and wellness flowchart 200 as described above for the favor and wellness program 120A, 120B.

The monitoring flowchart 300 may be used in combination with the favor and wellness flowchart 200 as a wellness application to monitor the group of two of more users for possible changes in their health. The monitoring flowchart 300 may identify at risk users and users who may need help. This may help the group of two or more users to help each other and identify those users who may have unanswered help or favors. Step 302 includes monitoring a first subset of the group of two or more users for at risk behavior.

At step 304, the favor and wellness program 120A, 120B identifies an at risk user as a user of the group of two or more users who have over a second threshold amount or percentage of favor requests which are unfulfilled. For example, the second threshold may be 50% or an amount of 10 over a period of time. For a user who has 55% of their favor requests unfulfilled, or 15 unfulfilled requests over a period of time, the third user may be identified as an at risk user. The favor and wellness program 120A, 120B may identify at risk users as those users whom have a percentage change over a third threshold amount of a change in a number of favor requests. For example, a user may have between 10 and 15 favor requests per month over a period of 8 months. The third threshold may be 75%. Then in the 9^{th} month, the fourth user may have a change in the number of favor requests greater than the third threshold. For example, there may be only 1 favor request, or alternatively, have 50 favor requests, and either of these situations are a change greater than 75% than the average number of favor requests per month. This example may identify an at risk user.

At step 306, the favor and wellness program 120A, 120B contacts a first group of one or more friends of the at risk user with information identifying why the at risk user has been highlighted as potentially needing help. The first group of one or more friends may be identified by the user as their emergency contacts, or their friends, or the favor and wellness program 120A, 120B may identify those users who have been most frequently in contact with the at risk user. In an embodiment, information regarding the at risk user may be private and only shared with one or more users whom the at risk user has identified as an emergency contact. In an embodiment, the at risk user may receive information about whom they could contact for help and encourage the at risk user to ask for assistance.

At step 308, the favor and wellness program 120A, 120B provides to the first group suggested ways of helping the at risk user. Suggestions may include identifying any outstanding favor requests of the at risk user, and suggesting to the first group changes to their own preferences of available times and services in order to accommodate an outstanding favor request of the at risk user. Additionally, suggestions may include prioritizing any current or future favor request from the at risk user, and/or suggestions to contact the at risk user. A history of favor requests of the at risk user may be provided to the first group, with details regarding fulfillment. Suggestions may be to call, visit, email or otherwise contact the at risk user. The first group may work together to help the at risk user. In some cases, the favor and wellness program 120A, 120B may suggest outside help for the at risk user.

At step 310, the favor and wellness program 120A, 120B increases a priority of any favor requests from the at risk user such that those requests are highlighted, or listed first or otherwise distinguished from other favor requests.

At step 312, the favor and wellness program 120A, 120B performs on-going monitoring of favor request frequency and fulfillment of the at risk user, with results sent to the first group.

The favor and wellness program 120A, 120B provides a cognitive solution for users of a favor application to request help for tasks they are having trouble completing, and also may identify a user who may need help. Balancing work life can be difficult and often it is hard to ask for help. The favor and wellness program 120A, 120B may help by making it more routine to see others ask for help and make it easier to request help, and also to provide specific help when needed. Often people would like to help others, but are unsure of what specific help they can provide. The favor and wellness program 120A, 120B may help to improve a quality and balance of life, and increase a sense of community for the users. The favor and wellness program 120A, 120B improves computer functionality by allowing simultaneous instant connection between two users and assists in the users connecting and providing assistance to each other. A company may provide this application for employees to help the employees help each other and ultimately help the company with more satisfied employees with an additional tool to balance the many responsibilities of their lives.

It may be appreciated that FIGS. 2 and 3 provide only an illustration of an implementation and do not imply any limitations with regard to how different embodiments may be implemented. Many modifications to the depicted environments may be made based on design and implementation requirements. For example, steps and sub-steps may be performed concurrently to other steps or sub-steps.

Referring now to FIG. 4, a block diagram of components of a computing device, such as the client computer 102 or the server computer 104, of the system 100 of FIG. 1, in accordance with an embodiment of the present invention is shown. It should be appreciated that FIG. 4 provides only an illustration of an implementation and does not imply any limitations with regard to the environments in which different embodiments may be implemented. Many modifications to the depicted environment may be made.

The computing device may include one or more processors 402, one or more computer-readable RAMs 404, one or more computer-readable ROMs 406, one or more computer readable storage media 408, device drivers 412, read/write drive or interface 414, network adapter or interface 416, all interconnected over a communications fabric 418. Communications fabric 418 may be implemented with any architecture designed for passing data and/or control information between processors (such as microprocessors, communications and network processors, etc.), system memory, peripheral devices, and any other hardware components within a system.

One or more operating systems 410, and one or more application programs 411, for example, the favor and wellness monitoring program 120A, 120B, are stored on one or more of the computer readable storage media 408 for execution by one or more of the processors 402 via one or more of the respective RAMs 404 (which typically include cache memory). In the illustrated embodiment, each of the computer readable storage media 408 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

The computing device may also include a R/W drive or interface 414 to read from and write to one or more portable computer readable storage media 426. Application programs 411 on the computing device may be stored on one or more of the portable computer readable storage media 426, read via the respective R/W drive or interface 414 and loaded into the respective computer readable storage media 408.

The computing device may also include the network adapter or interface 416, such as a TCP/IP adapter card or wireless communication adapter (such as a 4G wireless communication adapter using OFDMA technology). Application programs 411 on the computing device may be downloaded to the computing device from an external computer or external storage device via a network (for example, the Internet, a local area network or other wide area network or wireless network) and network adapter or interface 416. From the network adapter or interface 416, the programs may be loaded onto computer readable storage media 408. The network may comprise copper wires, optical fibers, wireless transmission, routers, firewalls, switches, gateway computers and/or edge servers.

The computing device may also include a display screen 420, a keyboard or keypad 422, and a computer mouse or touchpad 424. Device drivers 412 interface to display screen 420 for imaging, to keyboard or keypad 422, to computer mouse or touchpad 424, and/or to display screen 420 for pressure sensing of alphanumeric character entry and user selections. The device drivers 412, R/W drive or interface 414 and network adapter or interface 416 may comprise hardware and software (stored on computer readable storage media 408 and/or ROM 406).

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics of cloud computing include on-demand self-service, broad network access, resource pooling, rapid elasticity, and measured service, which are each described below.

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models include Software as a Service, Platform as a Service, and Infrastructure as a Service, which are each described below.

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models include private cloud, community cloud, public cloud, and hybrid cloud, which are each described below.

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 5, illustrative cloud computing environment 500 is depicted. As shown, cloud computing environment 500 includes one or more cloud computing nodes 510 with which local computing devices used by cloud consumers, such as, for example, personal digital assistant (PDA) or cellular telephone 540A, desktop computer 540B, laptop computer 540C, and/or automobile computer system 540N may communicate. Cloud computing nodes 510 may communicate with one another. They may be grouped (not shown) physically or virtually, in one or more networks, such as Private, Community, Public, or Hybrid clouds as described hereinabove, or a combination thereof. This allows cloud computing environment 500 to offer infrastructure, platforms and/or software as services for which a cloud consumer does not need to maintain resources on a local computing device. It is understood that the types of computing devices 540A-N shown in FIG. 5 are intended to be illustrative only and that cloud computing nodes 510 and cloud computing environment 500 can communicate with any type of computerized device over any type of network and/or network addressable connection (e.g., using a web browser).

Referring now to FIG. 6, a set of functional abstraction layers provided by cloud computing environment 500 (as shown in FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 660 includes hardware and software components. Examples of hardware components include: mainframes 661; RISC (Reduced Instruction Set Computer) architecture based servers 662; servers 663; blade servers 664; storage devices 665; and networks and networking components 666. In some embodiments, software components include network application server software 667 and database software 668.

Virtualization layer 670 provides an abstraction layer from which the following examples of virtual entities may be provided: virtual servers 671; virtual storage 672, for example the data storage device 110 as shown in FIG. 1; virtual networks 673, including virtual private networks; virtual applications and operating systems 674; and virtual clients 675.

In an example, management layer 680 may provide the functions described below. Resource provisioning 681 provides dynamic procurement of computing resources and other resources that are utilized to perform tasks within the cloud computing environment. Metering and Pricing 682 provide cost tracking as resources are utilized within the cloud computing environment, and billing or invoicing for consumption of these resources. In an example, these resources may include application software licenses. Security provides identity verification for cloud consumers and tasks, as well as protection for data and other resources. User portal 683 provides access to the cloud computing environment for consumers and system administrators. Service level management 684 provides cloud computing resource allocation and management such that required service levels are met. Service Level Agreement (SLA) planning and fulfillment 685 provide pre-arrangement for, and procurement of, cloud computing resources for which a future requirement is anticipated in accordance with an SLA.

Workloads layer 690 provides examples of functionality for which the cloud computing environment may be utilized. Examples of workloads and functions which may be provided from this layer include: mapping and navigation 691; software development and lifecycle management 692; virtual classroom education delivery 693; data analytics processing 694; transaction processing 695; and favor and wellness determination 696. The favor and wellness determination 696 may relate communicating favor requests between users and monitoring the wellness of each communicating user.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A method for favor monitoring, the method comprising:
sharing a first favor request from a first user to a first group of one or more users, wherein the favor request comprises a favor type, a favor time request start, a favor time request end, and one or more favor parameters;
receiving a first favor proposal from a second user of the first group;
receiving a first favor acknowledgement from the first user in response to the first favor proposal;
receiving confirmation of completion of the first favor;
documenting completion of the first favor, wherein documentation comprises the first favor request, the first user, and the second user;
monitoring a favor request history of a third user of the first group; and
identifying the third user as an at risk user based on a change greater than a first threshold amount of a number of favor requests over a period of time, and based on a percentage of unfulfilled requests greater than a second threshold amount.

2. The method according to claim 1, wherein the one or more favor parameters are selected from a group consisting of a requester name, a reason for the request, a type of request, a transaction type, a level of complexity, a date, and a location.

3. The method according to claim 1, wherein the favor type is selected from a group consisting of providing transportation, childcare, dog sitting, house sitting, picking up items, yard work, house work, borrow something, advice, need a place to stay, home maintenance, car maintenance, and recommendations.

4. The method according to claim 1, further comprising:
receiving a first favor proposal from the second user.

5. The method according to claim 1, further comprising:
collecting a user profile for each user of the first group, wherein a user profile may include at least one of a userid, a user name, a user email, a user address, a user phone number, a user availability calendar, or a user favor grant type.

6. The method according to claim 1, further comprising:
contacting an emergency contact user of the third user;
providing a favor request history to the emergency contact user; and
providing suggested actions to the third user.

7. The method according to claim 1, further comprising:
prioritizing favor requests of the at risk user.

8. A computer program product for favor monitoring, the computer program product comprising:
one or more computer-readable storage media and program instructions stored on the one or more computer-readable storage media, the program instructions comprising:
program instructions to share a first favor request from a first user to a first group of one or more users, wherein the favor request comprises, a favor type, a favor time request start, a favor time request end, and one or more favor parameters;
program instructions to receive a first favor proposal from a second user of the first group;
program instructions to receive a first favor acknowledgement from the first user in response to the first favor proposal;
program instructions to receive confirmation of completion of the first favor;
program instructions to document completion of the first favor, wherein documentation comprises the first favor request, the first user, and the second user;
program instructions to monitor a favor request history of a third user of the first group; and
program instructions to identify the third user as an at risk user based on a change greater than a first threshold amount of a number of favor requests over a period of time, and based on a percentage of unfulfilled requests greater than a second threshold amount.

9. The computer program product according to claim 8, wherein the one or more favor parameters are selected from a group consisting of a requester name, a reason for the request, a type of request, a transaction type, a level of complexity, a date, and a location.

10. The computer program product according to claim 8, wherein the favor type is selected from a group consisting of providing transportation, childcare, dog sitting, house sitting, picking up items, yard work, house work, borrow something, advice, need a place to stay, home maintenance, car maintenance, and recommendations.

11. The computer program product according to claim 8, further comprising:
program instructions to receive a first favor proposal from the second user.

12. The computer program product according to claim 8, further comprising:
program instructions to collect a user profile for each user of the first group, wherein a user profile may include at least one of a userid, a user name, a user email, a user address, a user phone number, a user availability calendar, a user favor grant type.

13. The computer program product according to claim 8, further comprising:
program instructions to contact an emergency contact user of the third user;
program instructions to provide a favor request history to the emergency contact user; and
program instructions to provide suggested actions to the third user.

14. The computer program product according to claim 8, further comprising:
program instructions to prioritize favor requests of the at risk user.

15. A computer system for favor monitoring, the computer system comprising:
one or more computer processors, one or more computer-readable storage media, and program instructions stored on the one or more of the computer-readable storage media for execution by at least one of the one or more processors, the program instructions comprising:
program instructions to share a first favor request from a first user to a first group of one or more users, wherein the favor request comprises, a favor type, a favor time request start, a favor time request end, and one or more favor parameters;
program instructions to receive a first favor proposal from a second user of the first group;
program instructions to receive a first favor acknowledgement from the first user in response to the first favor proposal;
program instructions to receive confirmation of completion of the first favor;
program instructions to document completion of the first favor, wherein documentation comprises the first favor request, the first user, and the second user;
program instructions to monitor a favor request history of a third user of the first group; and
program instructions to identify the third user as an at risk user based on a change greater than a first threshold amount of a number of favor requests over a period of time, and based on a percentage of unfulfilled requests greater than a second threshold amount.

16. The computer system according to claim 15, wherein the one or more favor parameters are selected from a group consisting of a requester name, a reason for the request, a type of request, a transaction type, a level of complexity, a date, and a location.

17. The computer system according to claim 15, wherein the favor type is selected from a group consisting of providing transportation, childcare, dog sitting, house sitting, picking up items, yard work, house work, borrow something, advice, need a place to stay, home maintenance, car maintenance, and recommendations.

18. The computer system according to claim 15, further comprising:
program instructions to receive a first favor proposal from the second user.

19. The computer system according to claim 15, further comprising:
program instructions to collect a user profile for each user of the first group, wherein a user profile may include at least one of a userid, a user name, a user email, a user address, a user phone number, a user availability calendar, a user favor grant type.

20. The computer system according to claim 15, further comprising:
program instructions to contact an emergency contact user of the third user;
program instructions to provide a favor request history to the emergency contact user; and
program instructions to provide suggested actions to the third user.
